(19) United States
(12) Patent Application Publication
(10) Pub. No.: US 2020/0237710 A1
(43) Pub. Date: Jul. 30, 2020

(54) SYNERGISTIC COMPOSITION OF VITAMIN C AND VITAMIN K1, AND COMPOSITIONS THEREOF FOR THE TREATMENT OF CANCER

(71) Applicant: ALFRED ORDMAN, ROCKFORD, IL (US)

(72) Inventor: ALFRED ORDMAN, ROCKFORD, IL (US)

(21) Appl. No.: 16/750,952

(22) Filed: Jan. 23, 2020

Related U.S. Application Data
(60) Provisional application No. 62/797,096, filed on Jan. 25, 2019.

(51) Int. Cl. A61K 31/341; A61K 31/122; A61K 33/24

(52) U.S. Cl. CPC A61K 31/341; A61K 33/24; A61K 31/122

(57) ABSTRACT

The present invention relates to a pharmaceutical composition comprising vitamin C, or pharmaceutically acceptable salt, solvate or hydrate thereof, in combination with vitamin K1, or pharmaceutically acceptable salt, solvate or hydrate thereof, and may or may not include chromium, or a pharmaceutically acceptable salt, solvate, or hydrate thereof and a pharmaceutically acceptable carrier. Further provided herein is the method of treating, managing, or preventing cancer in human comprising administering a predetermined dosage of pharmaceutical composition comprising Vitamin C or pharmaceutically acceptable salts thereof and Vitamin K1 or a pharmaceutically acceptable salt thereof and may or may not include chromium, or a pharmaceutically acceptable salt, solvate, or hydrate thereof at a predetermined time.

### TECHNICAL FIELD OF THE INVENTION

The present invention generally relates to the field of biochemistry and medicines, utilizing combination therapy to treat cancer. In particular the invention relates to use of pharmaceutical composition comprising vitamin C or a pharmaceutically acceptable salt, solvate, or hydrate thereof, in combination with vitamin K1 or a pharmaceutically acceptable salt, solvate, or hydrate thereof in managing, treating or preventing cancer in humans.

### BACKGROUND OF THE INVENTION

Cancer is one of the most widespread diseases affecting mankind, and a leading cause of death worldwide. In the United States alone, cancer is the second leading cause of death, surpassed only by heart disease. Cancer is often characterized by deregulation of normal cellular processes or unregulated cell proliferation. Cells that have been transformed to cancerous cells tend to proliferate in an uncontrolled and unregulated manner leading to, in some cases, metastasis or the spread of the cancer. Deregulation of the cell proliferation could result from the modification to one or more genes, responsible for the cellular pathways that control cell-cycle progression. Or it could result from DNA modifications (including but not limited to mutations, amplifications, rearrangements, deletions, and epigenetic gene silencing) in one or more cell-cycle checkpoint regulators which allow the cell to move from one phase of the cell cycle to another unchecked. Cancer is now primarily treated with one or more combinations of three types of therapies: surgery, radiation, and chemotherapy. Surgery involves the bulk removal of diseased tissue. While surgery is sometimes effective in removing tumours located at certain sites, for example, in the breast, colon, and skin, it cannot be used in the treatment of tumours located in other areas, such as the backbone.

Colon cancer (colorectal cancer, CRC) is a major health concern worldwide due to its high prevalence and mortality rate. In developed countries, it is the third most common malignancy and the second most common cause of cancer-related death. Although advances in the treatment of CRC have been made a major impact on its management, many patients with advanced disease will eventually die as a result of cancer.

Bladder cancer is the fifth most common cancer in North America. Non-muscle-invasive bladder cancer is difficult to treat and up to 95% percent of patients receiving drugs infused into the bladder (intravesical agents) will experience recurrence of the cancer.

The treatment of polycystic diseases described in U.S. Pat. No. 9,744,152 consists of a composition comprising Vitamin C and Vitamin K without Chromium for the treatment of polycystic disease which is specifically related to kidney or liver disease.

The management of superficial bladder carcinoma using two grams BID oral dosage of vitamin C was described by Edward folk, Tracy M. Downs and Alfred Roc Ordman. The paper describes the usage of high concentration of vitamin C alone for two days which will generate maximum concentration of vitamin C in the bladder. The high concentration of Vitamin C which develops can be sufficient to kill cancer cells in the bladder.

Furthermore, it is unknown what specific amount of Vitamin C and Vitamin K1 in combination may be able to treat, or even improve upon and/or provide superior treatments for killing cancer cells.

At low concentrations of vitamin C, it acts as an anti-oxidant and thereby traps free radicals. When vitamin K3 is present with the vitamin C, it regenerates the vitamin C so that it produces free radicals. This amplifies the radical generation by vitamin C. Therefore, when the vitamin C is amplified by vitamin K, it will generate free radicals that are able to kill cancer cells without harming normal cells.

For the foregoing reasons, there is a need to develop the best, most effective composition and dosing regimen that will most effectively treat cancer while minimizing undesired side effects and patient non-compliance with the medication. In addition, there is a need to improve upon the current available treatments or provide better, more efficacious treatment schedules, and/or more convenient or easier to comply with treatment regimens, for cancer patients.

Based on above, there is a requirement in developing pharmaceutical compositions for the treatment of cancers which is safe and acceptable according to FDA requirements.

The abovementioned shortcomings, disadvantages and problems are addressed herein, which will be understood by reading and studying the following specification.

### SUMMARY OF THE INVENTION

The present invention relates to a pharmaceutical composition comprising: (i) Vitamin C or a pharmaceutically acceptable salt thereof; and (ii) Vitamin K1 or a pharmaceutically acceptable salt thereof.

Another embodiment of the invention relates to a pharmaceutical composition comprising: (i) Vitamin C or a pharmaceutically acceptable salt thereof; and (ii) Vitamin K1 or a pharmaceutically acceptable salt thereof; and (iii) chromium or a pharmaceutically acceptable salt thereof; and a pharmaceutically acceptable carrier.

Yet another embodiment of the invention relates to a method of managing, treating or preventing cancer in human comprising administering a predetermined dosage of pharmaceutical composition comprising Vitamin C or pharmaceutically acceptable salts thereof and Vitamin K1 or a pharmaceutically acceptable salt thereof at a predetermined time.

Yet another embodiment of the present invention relates to a predetermined dosage of pharmaceutical composition comprising: (i) Vitamin C or a pharmaceutically acceptable salt thereof; and (ii) Vitamin K1 or a pharmaceutically acceptable salt thereof, and a pharmaceutically acceptable carrier.

Yet another embodiment of the present invention also relates to a dosage regimen given to the patient using pharmaceutical composition comprising: (i) Vitamin C or a pharmaceutically acceptable salt thereof; (ii) Vitamin K1 or a pharmaceutically acceptable salt thereof, and (iii) chromium or a pharmaceutically acceptable salt thereof, wherein the Vitamin C is in the range of 100 to 2000 mg, Vitamin K1 in the range of 0.1 to 100 mg, and chromium in the range of 12.5 to 450 mcg for every 12 hours for 2 consecutive days in a month.

Yet another embodiment of the present invention relates to the pharmaceutical composition comprising predetermined dosage of vitamin C, or a pharmaceutically acceptable salt, solvate, or hydrate thereof in combination with vitamin K1, or a pharmaceutically acceptable salt, solvate, or hydrate thereof, when administered to the subject or patient, the higher concentration of vitamin C present in the combination enhances the ability to generate free radicals and thereby kills the cancerous cells. Further the vitamin K1 is metabolized in the subject to produce vitamin K2 and intermediately vitamin K3, wherein the vitamin K3 reacts with the vitamin C thereby amplifies the radical generating capacity of vitamin C by up to 40-fold, which is sufficient to kill the cancerous cells without harming the normal cells. Therefore, at higher concentration vitamin C acts as a pro-oxidant. Furthermore, at low concentrations of vitamin C it acts as an anti-oxidant and thereby traps the free radicals. In addition, an intake of vitamin K1 far above the daily value of 90 mcg will activate decalcification enzymes, which reduces hardening of the arteries and heart damage, thus reducing the heart disease.

Yet another embodiment of the present invention relates to the pharmaceutical composition comprising predetermined dosage of vitamin C, or a pharmaceutically acceptable salt, solvate, or hydrate thereof in combination Chromium with vitamin K1, or a pharmaceutically acceptable salt, solvate, or hydrate thereof when administered to the subject or patient, the higher concentration of vitamin C present in the combination enhances the ability to generate free radicals and thereby kills the cancerous cells. Chromium in the range of 12.5 meg to 450 mcg has been added to address various health conditions which are likely to contribute to development of cancer. Among the most active areas of chromium research are its use in supplement form to treat diabetes, lower blood lipid levels, promote weight loss, and improve body composition [ref3]. Diabetes and obesity both contribute to the development of cancer. To obtain better performance of our invention to kill cancer cells and prevent cancer development, we include chromium.

However, the human consumption of vitamin K3 is not allowed in US and hence vitamin K3 is not available commercially. Therefore, the present invention utilizes the vitamin K1 which is metabolized in humans to produce vitamin K2, and intermediately to vitamin K3. Thus, the vitamin K3 in combination with vitamin C regenerates the capacity of vitamin C to produce free radicals, thereby killing the cancerous cells without harming the normal cells. Therefore, one can safely consume the pharmaceutical composition comprising the vitamin C in combination with vitamin K1 thereby enhancing the free radicals in the blood of the patients and killing the cancerous cells, while taking chromium reduces the likelihood that those cancer cells will even form. Hence the present invention is the safest and an inexpensive way to prevent and halt cancer in its earliest development.

### DETAILED DESCRIPTION OF THE INVENTION

The present invention relates to a pharmaceutical composition and method of managing, treating or preventing cancer. In particular, the present invention relates to a pharmaceutical composition comprising combination of vitamin C or a pharmaceutically acceptable salt, solvate, or hydrate thereof, and vitamin K1 or a pharmaceutically acceptable salt, solvate or hydrate thereof, in managing, treating or preventing cancer in humans.

Before the present invention is disclosed and described, it is to be understood that this invention is not limited to the particular process steps and materials disclosed herein, as such process steps and materials may vary to some degree. It is also to be understood that the terminology used herein is used for the purpose of describing particular embodiments only and is not intended to be limiting as the scope of the present invention will be limited only by appended claims and equivalents thereof.

In order to more clearly and concisely describe and point out the subject matter of the claimed invention, the following definitions are provided for specific terms which are used in the following written description.

The terms “cancer” and “cancerous” when used herein refer to or describe the physiological condition in subjects that is typically characterized by unregulated cell growth. Examples of cancer include, without limitation, carcinoma, lymphoma, sarcoma, blastoma and leukaemia. More particular examples of such cancers include squamous cell carcinoma, lung cancer, pancreatic cancer, cervical cancer, ovarian cancer, uterine cancer, bladder cancer, hepatoma, breast cancer, colon carcinoma, and head and neck cancer. While the term “cancer” as used herein is not limited to any one specific form of the disease, it is believed that the combination treatment methods provided by the invention will be particularly effective for a variety of cancers in a subject in need of treatment. It is particularly useful as soon as cancer cells appear in the body, prior to a human or physician being aware of or able to detect the beginning of cancer.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “Vitamin C” also termed as ascorbic acid, Acide Ascorbique, Acide Cévitamique, Acide Iso-Ascorbique, Acide L-Ascorbique, AcidoAscorbico, Antiscorbutic Vitamin, Ascorbate, Ascorbate de Calcium, Ascorbate de Sodium, Ascorbic Acid, Ascorbic acid, Ascorbyl Palmitate, Calcium Ascorbate, Cevitamic Acid, Iso-Ascorbic Acid, L-Ascorbic Acid, Magnesium Ascorbate or dehydroascorbate.

The term “Vitamin K1” also known as Phytonadione; 84-80-0; Phytomenadione; Phylloquinone; 3-Phytylmenadione.

The term “pharmaceutically acceptable salt” includes treating required compound with corresponding acid or base accordingly to form a corresponding pharmaceutically acceptable salt which retain the biological effectiveness and properties of the active ingredient of the biochemical composition, which are not otherwise undesirable. Pharmaceutically acceptable salts include, but are not limited to, salts of sodium, potassium, calcium, magnesium, aluminium and the like.

The term “effective amount” refers to that an amount of a composition of the disclosure that when administered to an individual subject in need thereof, is sufficient to reduce the cancer cell activity and/or growth thereby enhancing the anticancer activity.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “manage,” “managing,” and “management” refer to preventing or slowing the progression, spread, or worsening of a condition, disorder, or disease, or of one or more symptoms (e.g., pain) thereof. Sometimes, the beneficial effects that a subject derives from a prophylactic or therapeutic agent do not result in a cure of the condition, disorder, or disease. In one embodiment, the term management refers to preventing or slowing the progression, spread, or worsening of the pain of the cancer.

The terms “therapeutically effective amount” and “effective amount” are meant to include the amount of a compound or combination of compounds that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease.

According to one embodiment of the present invention, it provides a pharmaceutical composition comprising vitamin C or a pharmaceutically acceptable salt, solvate, or hydrate thereof in combination with vitamin K1, or a pharmaceutically acceptable salt, solvate, or hydrate thereof. In another embodiment, the pharmaceutical compositions provided herein further comprise a pharmaceutically acceptable vehicle, carrier, diluent, or excipient, or a mixture thereof.

Yet another embodiment of the present invention provides a method of treating, managing or preventing cancer in a subject by administrating of a predetermined dose of a therapeutically effective amount of vitamin C, or a pharmaceutically acceptable salt, solvate, or hydrate thereof in combination with vitamin K1, or a pharmaceutically acceptable salt, solvate, or hydrate thereof to the subject at a predetermined time.

According to another embodiment of the present invention, it provides a pharmaceutical composition comprising vitamin C or a pharmaceutically acceptable salt, solvate, or hydrate thereof in combination with vitamin K1, or a pharmaceutically acceptable salt, solvate, or hydrate thereof, and chromium or a pharmaceutically acceptable salt thereof, and a pharmaceutically acceptable carrier. In another embodiment, the pharmaceutical compositions provided herein further comprise a pharmaceutically acceptable vehicle, carrier, diluent, or excipient, or a mixture thereof.

Yet another embodiment of the present invention provides a method of treating, managing or preventing cancer in a subject by administrating of a predetermined dose of a therapeutically effective amount of vitamin C, or a pharmaceutically acceptable salt, solvate, or hydrate thereof in combination with vitamin K1, or a pharmaceutically acceptable salt, solvate, or hydrate thereof, and chromium or a pharmaceutically acceptable salt thereof, and a pharmaceutically acceptable carrier, to the subject at a predetermined time.

According to one embodiment herein, vitamin C refers to ascorbic acid or a pharmaceutically acceptable salt thereof; or a pharmaceutically acceptable solvate or hydrate thereof. Vitamin C is also known as xyloascorbic acid, 3-oxo-L-gulofuranolactone (enol form), L-3-ketothreohexuronic acid lactone, antiscorbutic vitamin, cevitamic acid, adenex, allercorb, ascorin, ascorteal, ascorvit, cantan, cantaxin, catavin C, cebicure, cebion, cecon, ocegiolan, celaskon, celin, cenetone, cereon, cergona, cescorbat, cetamid, cetabe, cetemican, ccvalin, cevatine, cevex, cevimin, ce-yl-sol, cevitan, cevitex, cewin, ciamin, cipca, concemin, C-vin, daviamon C, duoscorb, hybrin, laroscorbine, lemascorb, planavit C, proscorbin, redoxon, ribena, scorbacid, scorbu-C, testascorbic, vicelat, vitaceec, vitacimin, vitacin, vitascorbol, and xitix.

According to one embodiment herein, the vitamin C is ascorbic acid. In another embodiment, vitamin C provided herein is a pharmaceutically acceptable salt of ascorbic acid, or pharmaceutically acceptable solvate or hydrate thereof. Further the suitable bases used in the preparation of pharmaceutically acceptable salts, includes but not limited to inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

According to one embodiment herein, vitamin C is an alkali or alkaline earth metal salt of ascorbic acid, or a pharmaceutically acceptable solvate or hydrate thereof. In another embodiment, vitamin C provided herein is sodium, potassium, calcium, or magnesium ascorbate, or a pharmaceutically acceptable solvate or hydrate thereof. In yet another embodiment, vitamin C provided herein is sodium ascorbate, or a pharmaceutically acceptable solvate or hydrate thereof. In yet another embodiment, vitamin C provided herein is sodium ascorbate, which is also known as vitamin C sodium, ascorbin, sodascorbate, natrascorb, cenolate, ascorbicin, or cebitate. In yet another embodiment, vitamin C provided herein is potassium ascorbate, or a pharmaceutically acceptable solvate or hydrate thereof. In yet another embodiment, vitamin C provided herein is magnesium ascorbate, or a pharmaceutically acceptable solvate or hydrate thereof. In still another embodiment, vitamin C provided herein is calcium ascorbate or a pharmaceutically acceptable solvate or hydrate thereof.

According to one embodiment herein, vitamin C is selected from any biologically acceptable form of an ascorbic acid, ascorbyl ester or ascorbate including either or both water-soluble and fat-soluble forms. The water-soluble form of ascorbic acid can be selected from the group consisting of ascorbic acid, a biologically acceptable mono or divalent metal ion salt of ascorbic acid and niacinamide ascorbate, and mixtures thereof. Furthermore, the other water-soluble forms of ascorbic acid include, but are not limited to manganese ascorbate; zinc ascorbate; iron ascorbate; copper ascorbate; boron ascorbate; molybdenum ascorbate; and chromium ascorbate. Furthermore, the fat-soluble ascorbyl esters preferably comprise fatty acid esters of saturated or unsaturated carboxylic acids with ascorbyl palmitate being the preferred form. Besides, the other fat-soluble esters of ascorbic acid include, but are not limited to ascorbyl palmitate; ascorbyl arachidonate; ascorbyl stearate; ascorbyl linoleate; ascorbyl linoleneate; and ascorbyl oleate.

According to one embodiment herein, the vitamin K1, or pharmaceutically acceptable salt, solvate or hydrate thereof refers to phytonadione, phylloquinone, [R—[R*,R*-(E)]]-2-methyl-3-(3,7,11,15-tetramethyl-2-hexadecenyl)-1,4-naphthalenedione, 2-methyl-3-phytyl-1,4-naphthoquinone, 3-phytylmenadione, phytomenadione, phytonadione, aqua-merphyton, konakion, mephyton, mono-day, veda-K1, and veta-K1.

According to one embodiment herein, the pharmaceutical composition comprising vitamin C and vitamin K1 is formulated together in an oral dosage form and administered orally into the subject. In yet another embodiment of the present invention the pharmaceutical composition comprising vitamin C and vitamin K1 can be packaged separately and administered orally in predetermined oral dosage form. In yet another embodiment of the present invention the suitable oral dosage form includes, but not limited to tablet, pill, powder, capsule, troche, caplet, effervescent or non-effervescent powders or granules, lozenges and pellets. Furthermore, in addition to the active ingredient(s) vitamin C and vitamin K1, the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

According to another embodiment herein, the pharmaceutical composition comprising vitamin C, vitamin K1 and chromium is formulated together in an oral dosage form and administered orally into the subject. In yet another embodiment of the present invention the pharmaceutical composition comprising vitamin C, vitamin K1 and chromium can be packaged separately and administered orally in predetermined oral dosage form. In yet another embodiment of the present invention the suitable oral dosage form includes, but not limited to tablet, pill, powder, capsule, troche, caplet, effervescent or non-effervescent powders or granules, lozenges and pellets. Furthermore, in addition to the active ingredient(s) vitamin C, vitamin K1 and chromium, the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Consequently, the binders or granulators impart cohesiveness to a tablet to ensure the tablet remains intact after compression. Hence, the suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Further, the suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. Besides the amount of a binder or filler in the pharmaceutical compositions provided herein varies upon the type of formulation, and is readily discernible to those of ordinary skill in the art.

Moreover, the suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Hence, such compressed tablets can be used as chewable tablets. Therefore, the amount of a diluent in the pharmaceutical compositions provided herein varies upon the type of formulation, and is readily discernible to those of ordinary skill in the art.

Besides, the suitable disintegrants include, but are not limited to, agar, bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. Further the amount of a disintegrant in the pharmaceutical compositions provided herein varies upon the type of formulation, and is readily discernible to those of ordinary skill in the art.

In addition the suitable lubricants include but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar, starch; lycopodium; silica or silica gels, such as AEROSIL® 200 (W.R. Grace Co., Baltimore, Md.) and CAB-O-SIL® (Cabot Co. of Boston, Mass.); and mixtures thereof.

According to one embodiment herein, the pharmaceutical composition comprising vitamin C or a pharmaceutically acceptable salt, solvate, or hydrate thereof in combination with vitamin K1 or a pharmaceutically acceptable salt, solvate, or hydrate thereof as active ingredient (s) is administered in to the subject every 12 hours for 2 consecutive days more or less but preferably twice in a month.

According to one embodiment herein, the predetermined dosage of vitamin C or a pharmaceutically acceptable salt, solvate, or hydrate thereof is present in the range of 100 mg to 2,000 mg. Furthermore, the predetermined dosage of vitamin K or a pharmaceutically acceptable salt, solvate, or hydrate thereof is present in the range of 0.1 mg to 100 mg respectively.

According to one embodiment herein, the pharmaceutical composition comprising vitamin C or a pharmaceutically acceptable salt, solvate, or hydrate thereof in combination with vitamin K1 or a pharmaceutically acceptable salt, solvate, or hydrate thereof and chromium or a pharmaceutically acceptable salt, solvate, or hydrate thereof as active ingredient (s) is administered in to the subject every 12 hours for 2 consecutive days more or less but preferably twice in a month.

According to one embodiment herein, the predetermined dosage of vitamin C or a pharmaceutically acceptable salt, solvate, or hydrate thereof is present in the range of 100 mg to 2,000 mg. Furthermore, the predetermined dosage of vitamin K1 or a pharmaceutically acceptable salt, solvate, or hydrate thereof is present in the range of 0.1 mg to 100 mg respectively. Also, the predetermined dosage of chromium or a pharmaceutically acceptable salt, solvate, or hydrate thereof is in the range of 12.5 to 450 mcg.

According to one embodiment herein, the cancer includes, but not limited to head and neck cancer (e.g., originating from lip, oral cavity, oropharynx, hypopharynx, larynx, nasopharynx, nasal cavity, paranasal sinuses, or salivary glands), lung cancer (including small cell lung cancer and non-small cell lung cancer), gastrointestinal tract cancer (including esophageal cancer), gastric cancer, colorectal cancer, anal cancer, pancreatic cancer, liver cancer, gallbladder cancer, kidney cancer, skin cancer, extrahepatic bile duct cancer, cancer of the ampulla of vater, breast cancer, gynaecologic cancer (including cancer of uterine cervix, cancer of the uterine body, vaginal cancer, vulvar cancer, ovarian cancer, and gestational trophoblastic cancer neoplasia), testicular cancer, urinary tract cancer (including renal cancer), urinary bladder cancer, prostate cancer, penile cancer, urethral cancer, neurologic tumour, endocrine neoplasms (including carcinoid and islet cell tumour), pheochromocytoma, adrenal cortical carcinoma, parathyroid carcinoma and metastases to endocrine glands, lymphomas, Burkitt lymphoma, and Zollinger-Ellision syndrome. In certain embodiments, the proliferative disease cancer is a solid tumour.

According to one embodiment herein, the pharmaceutical composition comprising predetermined dosage of vitamin C, or a pharmaceutically acceptable salt, solvate, or hydrate thereof in combination with vitamin K1, or a pharmaceutically acceptable salt, solvate, or hydrate thereof, when administered to the subject or patient, the higher concentration of vitamin C present in the combination enhances the ability to generate free radicals and thereby kills the cancerous cells. Further the vitamin K1 is metabolized in the subject to produce vitamin K2 and intermediately vitamin K3, wherein the vitamin K3 reacts with the vitamin C thereby amplifies the radical generating capacity of vitamin C by at least 20-fold, which is sufficient to kill the cancerous cells without harming the normal cells. Therefore, at higher concentration vitamin C acts as a pro-oxidant. Furthermore, at low concentrations of vitamin C act as an anti-oxidant thereby traps the free radicals. In yet another embodiment of the present invention the predetermined dosage of vitamin C or a pharmaceutically acceptable salt, solvate, or hydrate thereof is present in the range of 100 mg to 2,000 mg. Furthermore, the predetermined dosage of vitamin K1 or a pharmaceutically acceptable salt, solvate, or hydrate thereof is present in the range of 0.1 mg to 100 mg respectively. In addition, an intake of vitamin K1 far above the daily value of 90 mcg will activate decalcification enzymes, which reduces hardening of the arteries and heart damage, thus reducing the heart disease.

From the foregoing discussion, it is apparent that the vitamin C or a pharmaceutically acceptable salt, solvate, or hydrate thereof when consumed in combination with vitamin K1 or a pharmaceutically acceptable salt, solvate, or hydrate thereof; the higher dose of vitamin C present in the composition enhances the ability of the vitamin C to generate free radicals, thereby killing the cancerous cells without harming the normal cells. Furthermore, the vitamin K1 is metabolized in the subject to produce vitamin K2 and thereby in the metabolic process vitamin K3. The vitamin K3 thus produced reacts with vitamin C regenerates the capacity of vitamin C to produce free radicals thereby killing the cancerous cells without harming the normal cells. However, the human consumption of vitamin K3 is not allowed in US and hence vitamin K3 is not available commercially. Therefore, the present invention utilizes the vitamin K1 which is metabolized in humans to produce vitamin K2, and intermediately to vitamin K3. Thus, the vitamin K3 in combination with vitamin C regenerates the capacity of vitamin C to produce free radicals, thereby killing the cancerous cells without harming the normal cells. Therefore, one can safely consume the pharmaceutical composition comprising the vitamin C in combination with vitamin K1 and thereby enhancing the free radicals in the blood of the patients and killing the cancerous cells. Hence the present invention is the safest and an inexpensive way to treat the cancer.

According to one embodiment herein, the pharmaceutical composition comprising predetermined dosage of vitamin C, or a pharmaceutically acceptable salt, solvate, or hydrate thereof in combination with vitamin K1, or a pharmaceutically acceptable salt, solvate, or hydrate thereof, and chromium, or a pharmaceutically acceptable salt, solvate, or hydrate thereof when administered to the subject or patient, the higher concentration of vitamin C present in the combination enhances the ability to generate free radicals and thereby kills the cancerous cells. Further the vitamin K1 is metabolized in the subject to produce vitamin K2 and intermediately vitamin K3, wherein the vitamin K3 reacts with the vitamin C thereby amplifies the radical generating capacity of vitamin C by at least 20-fold, which is sufficient to kill the cancerous cells without harming the normal cells. Therefore, at higher concentration vitamin C acts as a pro-oxidant. In yet another embodiment of the present invention the predetermined dosage of vitamin C or a pharmaceutically acceptable salt, solvate, or hydrate thereof is present in the range of 100 mg to 2,000 mg. Furthermore, the predetermined dosage of vitamin K1 or a pharmaceutically acceptable salt, solvate, or hydrate thereof is present in the range of 0.1 mg to 100 mg respectively. In addition, an intake of vitamin K1 far above the daily value of 90 mcg will activate decalcification enzymes, which reduces hardening of the arteries and heart damage, thus reducing the heart disease. Also, the intake of chromium or a pharmaceutically acceptable salt, solvate, or hydrate thereof in the range of 12.5 to 450 mcg will reduce the risk of developing conditions a subject which would increase the risk of developing cancer cells.

From the foregoing discussion, it is apparent that the vitamin C or a pharmaceutically acceptable salt, solvate, or hydrate thereof when consumed in combination with vitamin K1 or a pharmaceutically acceptable salt, solvate, or hydrate thereof; the higher dose of vitamin C present in the composition enhances the ability of the vitamin C to generate free radicals, thereby killing the cancerous cells without harming the normal cells. Furthermore, the vitamin K1 is metabolized in the subject to produce vitamin K2 and thereby in the metabolic process vitamin K3. The vitamin K3 thus produced reacts with vitamin C regenerates the capacity of vitamin C to produce free radicals thereby killing the cancerous cells without harming the normal cells. However, the human consumption of vitamin K3 is not allowed in US and hence vitamin K3 is not available commercially. Therefore, the present invention utilizes the vitamin K1 which is metabolized in humans to produce vitamin K2, and intermediately to vitamin K3. Thus, the vitamin K3 in combination with vitamin C regenerates the capacity of vitamin C to produce free radicals, thereby killing the cancerous cells without harming the normal cells. Therefore, one can safely consume the pharmaceutical composition comprising the vitamin C in combination with vitamin K1 and chromium, thereby enhancing the free radicals in the blood of the patients and killing the cancerous cells as well as preventing their initial occurrence. Hence the present invention is both a safe and an inexpensive way to prevent and treat the cancer.

What is claimed is:

1. A method of treating, preventing and managing cancer in a subject, comprising one of a 100 mg to 2,000 mg dose of vitamin C, or a pharmaceutically acceptable salt, solvate, or hydrate of Vitamin C, in combination with one of a 0.1 mg to 100 mg of vitamin K1, or a pharmaceutically acceptable salt, solvate, or hydrate of Vitamin K1 to the subject at a predetermined time which is at least once a month.

2. The method of claim 1, wherein the predetermined time is administering the combination of predetermined dosage of vitamin C and vitamin K1 every 12 hours for 2 consecutive days consumed at least once in a month.

3. The method of claim 1, wherein the cancer is one or more cancer cells of the prostate, ovary, endometrium, breast, bladder, colon, kidney, liver, lungs, esophagus, pancreas, stomach, uterus, cervix, head and neck, thyroid, brain or skin, or a combination thereof.

4. The method of claim 1, wherein the vitamin C and vitamin K1 are formulated together in an oral dosage form and administered orally.

5. The method of claim 1, wherein the oral dosage form is selected from the group consisting of tablet, pill, powder, capsule and the like.

6. The method of claim 1, wherein the vitamin C and vitamin K1 are packaged separately and taken in predetermined oral dosage form.

7. The method of claim 1, wherein vitamin C is ascorbic acid or a pharmaceutically acceptable salt thereof, or a pharmaceutically acceptable solvate or hydrate thereof.

8. The method of claim 1, wherein vitamin C is an alkali or alkaline earth metal salt of ascorbic acid, or a pharmaceutically acceptable solvate or hydrate thereof.

9. The method of claim 1, wherein vitamin C is sodium ascorbate, calcium ascorbate or magnesium ascorbate, or a pharmaceutically acceptable solvate or hydrate thereof.

10. The method of claim 1, wherein the vitamin K1 is phytonadione, phytomenadione; phylloquinone; 3-phytylmenadione, or a pharmaceutically acceptable salt thereof, or a pharmaceutically acceptable solvent or hydrate thereof.

11. A method of treating, preventing and managing cancer in a subject comprising pharmaceutical composition in a predetermined dose of therapeutically effective amount one of a 100 mg to 2,000 mg dose of vitamin C or a pharmaceutically acceptable salt, solvate, or hydrate of Vitamin C, in combination with one of a 0.1 mg to 100 mg of vitamin K1, or a pharmaceutically acceptable salt, solvate, or hydrate of vitamin K1, wherein the vitamin K1 of the said combination enhances the ability of the vitamin C to generate free radicals to kill the cancerous cells and vitamin K1 is metabolized in the subject to produce vitamin K2 and intermediately vitamin K3, wherein the vitamin K3 reacts with the vitamin C to amplify the radical generating capacity of vitamin C by up to 40 times which is sufficient to kill the cancerous cells.

12. The method of claim 12, wherein the radical generating capacity of Vitamin C to kill cancerous cells is increased by up to 40-fold.

13. The method of claim 12, wherein the subject is human.

14. A method of treating, preventing and managing cancer in a subject by administering to the subject, at a predetermined time which is at least once a month, a pharmaceutical composition in a predetermined dose of therapeutically effective amount of a 100 mg to 2,000 mg dose of vitamin C or a pharmaceutically acceptable salt, solvate, or hydrate of Vitamin C, in combination with 0.1 mg to 100 mg of vitamin K1, or a pharmaceutically acceptable salt, solvate, or hydrate of vitamin K1, wherein the vitamin K1 of the said combination enhances the ability of the vitamin C to generate free radicals to kill the cancerous cells and vitamin K1 is metabolized in the subject to produce vitamin K2 and intermediately vitamin K3, wherein the vitamin K3 reacts with the vitamin C to amplify the radical generating capacity of vitamin C sufficient to kill the cancerous cells, and in combination with a predetermined dosage from 12.5 mcg to 450 mcg of chromium or a pharmaceutically acceptable salt, solvate to reduce the risk of developing cancer cells.
